(19) United States
(12) Patent Application Publication
(10) Pub. No.: US 2008/0177385 A1
(43) Pub. Date: Jul. 24, 2008

(54) Testicular Prosthesis

(75) Inventor: Scott E. Jahns, Hudson, WI (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/016,535

(22) Filed: Jan. 18, 2008

Related U.S. Application Data
(60) Provisional application No. 60/885,542, filed on Jan. 18, 2007.

(51) Int. Cl. A61F 2/02

(52) U.S. Cl. 623/11.11

(57) ABSTRACT

A testicular prosthesis includes a flexible bladder, a fill port, and a plug. The flexible bladder incorporates an interior chamber. The fill port is coupled to the flexible bladder and generally includes a cylindrical tube that extends through the bladder. The cylindrical tube is defined by an interior wall and forms an opening to the interior chamber of the bladder. The plug is a cylindrical member that is coaxial to the bore. A seal is formed between the cylindrical member of the plug and the interior wall of the fill port.

### RELATED APPLICATIONS

The present application claims priority to U.S. provisional patent application No. 60/885,542, filed Jan. 18, 2007, and entitled “Testicular Prosthesis.” The identified provisional patent application is hereby incorporated by reference in its entirety.

### BACKGROUND OF THE INVENTION

The cosmetic affects of missing a testicle(s) either from congenital malformation, trauma or cancer treatment can impact the individuals physiological persona. A Testicular prosthesis is implanted in the scrotum of male patients to replicate a lost testicle for the purpose of offsetting the negative psychological effects that can stem from the absence of a testicle.

### SUMMARY OF THE INVENTION

Embodiments of the present invention are directed to testicular prosthesis that are implantable within a patient for the purpose of replacing a natural testicle. In one embodiment, the testicular prosthesis includes a flexible bladder having an interior chamber, a fill port coupled to the flexible bladder and a plug. The fill port includes a cylindrical tube extending through the bladder and have a cylindrical bore that is defined by an interior wall informs an opening to the interior chamber of the bladder. The plug includes a cylindrical member that is coaxial to the bore. A seal is formed between the cylindrical member of the plug and the interior wall of the fill port.

In accordance with another embodiment of the invention, the testicular prosthesis includes a flexible bladder having an interior chamber, the fill port plug receiver coupled to the flexible bladder and a fill port plug. The fill port plug receiver includes a housing having an open top, a base having an opening and cylindrical interior cavity defined by an interior wall. An o-ring is coupled to the base within the interior cavity and surrounds the opening. The fill port plug includes a cylindrical member received within the interior cavity of the fill port plug receiver and a fill port extending through the cylindrical member. The fill port plug is rotatable within the receiver between a fill position, in which the fill port is aligned with the opening in the base of the receiver, and a sealed position, in which the opening in the base of the receiver is sealed through engagement between the o-ring and a bottom surface of the cylindrical member.

### BRIEF DESCRIPTION OF THE DRAWINGS

FIG. 1 is an oblique view of a testicular prosthesis in accordance with embodiments of the invention.

FIG. 2 is an exploded cross-sectional view of the testicular prosthesis shown in FIG. 1 taken generally along line 2-2, in accordance with embodiments of the invention.

FIG. 3 is a partial cross-sectional view of a fluid-filling site of a testicular prosthesis in accordance with embodiments of the invention.

FIG. 3A is a variation on a partial cross-sectional view of a fluid-filling site of a testicular prosthesis in accordance with embodiments of the invention.

FIG. 3B is a variation on a partial cross-sectional view of a fluid-filling site of a testicular prosthesis in accordance with embodiments of the invention.

FIGS. 4 and 5 are exploded cross-sectional views of a filling site of a testicular prosthesis in accordance with embodiments of the invention.

FIG. 6 is a partial cross-sectional view of a filling site of a testicular prosthesis during a filling operation, in accordance with embodiments of the invention.

FIG. 7 is an exploded partial cross-sectional view of a filling site of a testicular prosthesis in accordance with embodiments of the invention.

### DETAILED DESCRIPTION OF ILLUSTRATIVE EMBODIMENTS

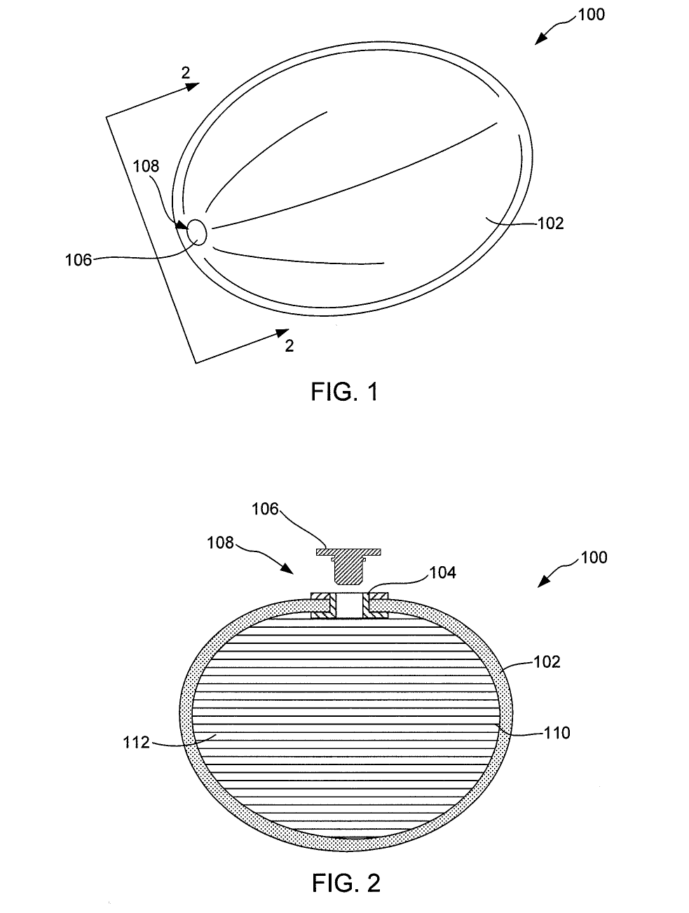

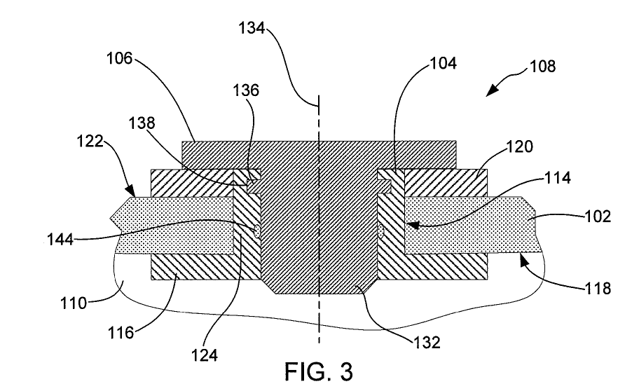

FIGS. 1 and 2 respectively show an oblique view of a testicular prosthesis 100 and a cross-sectional view of the testicular prosthesis 100 taken generally along line 2-2 of FIG. 1, in accordance with embodiments of the invention. One embodiment of the testicular prosthesis 100 includes a flexible bladder 102, a fill port 104 and a plug 106. The fill port 104 and the plug 106 are positioned at a filling site 108 of the testicular prosthesis 100. The plug 106 is shown as being exploded from the fill port 104 in FIG. 2.

The flexible bladder 102 has an interior chamber 110 that is filled with a bio-compatible solution 112, such as saline or a more viscous substance, such as a gel of hyaluronic acid, to expand the bladder 102 to an inflated state shown in FIGS. 1 and 2. The bladder 102 is configured to represent the male testicle when it is in the inflated state. Thus, in one embodiment, the bladder 102 has an oval or spherical inflated shape.

One embodiment of the bladder 102 is formed of a bio-compatible polymer generally possessing elastic properties, including various grades of silicone rubber, polyurethanes, PVC or other suitable material. The bladder can be formed through dip molding, injection molding, spray up, transfer molding, or other polymer processing method.

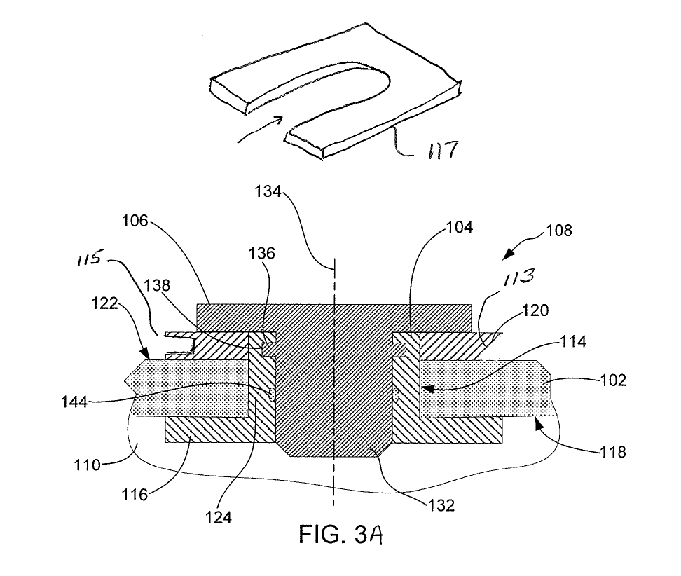

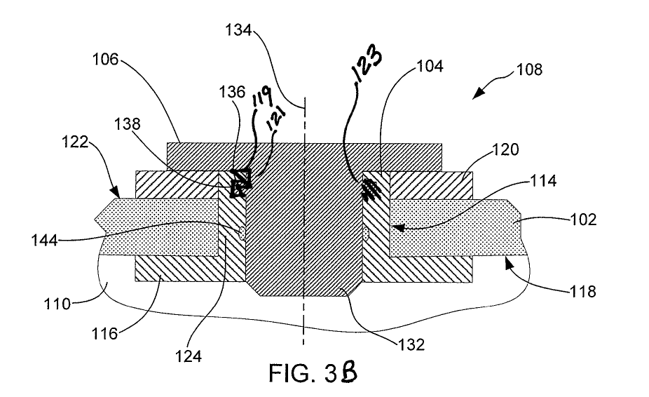

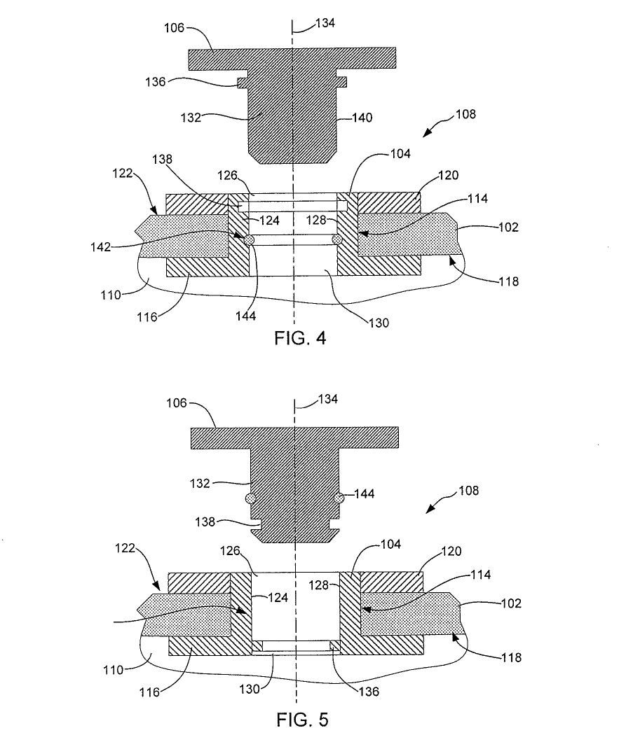

The fill port 104 is attached to the bladder 102 at an opening 114 in the bladder as best shown in FIGS. 3-5. A seal is formed between the bladder 102 and the fill port 104 to prevent the escape of fluid at that junction. One embodiment of the fill port 104 includes an annular collar 116 that is bonded to the bladder 102 using an adhesive, a weld, a chemical solvent, or other suitable method. In one embodiment, the collar 116 is bonded to an interior surface 118 of the bladder 102. In accordance with another embodiment, a retention ring 120 is coupled to the fill port 104 and/or an exterior surface 122 of the bladder 102. In one embodiment, the retention ring 120 opposes the collar 116, and the bladder 102 is pinched between the retention ring 120 and the collar 116 to secure the fill port 104 to the bladder 102 and seal the junction between the bladder 102 and the fill port 104.

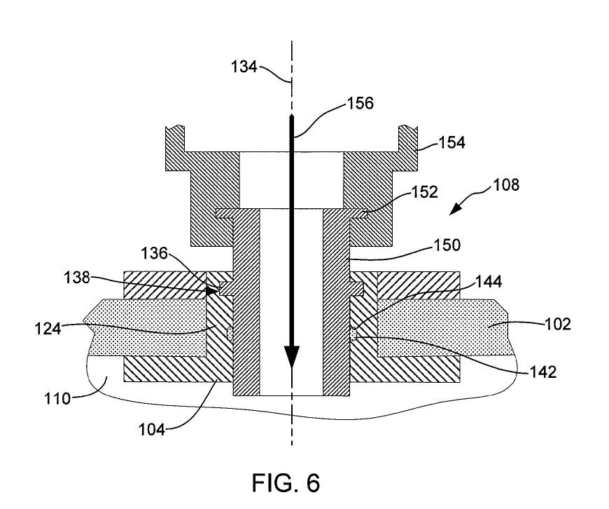

One embodiment of the fill port 104 includes a cylindrical tube 124 extending through the opening 114 in the bladder 102. The cylindrical tube 124 includes a bore 126 (FIG. 4) that is defined by an interior wall 128 of the tube 124 and provides an opening 130 to the interior chamber 110 of the bladder 102. The plug 106 comprises a cylindrical member 132 that is coaxial to a central access 134 of the bore 126 when the plug 106 is installed in the fill port 104, a shown in FIG. 3.

Embodiments of the invention relate to retaining the plug 106 within the fill port 104. Exemplary methods for retaining the plug 106 within the fill port 104 include providing cooperating threads on the cylindrical member 132 of the plug 106 and the interior wall 128 of the fill port 104 and screwing the plug 106 into the tube 124 of the fill port 104, gluing the plug 106 in the tube 124 of the fill port 104, welding the plug 106 to the fill port 104, and other suitable methods.

In order to utilize the methods described above for retaining the plug 106, a means of holding the prosthesis 100 without pushing fluid out of the prosthesis 100 is needed. FIG. 3A provides a bevel 113 and a slot 115 configuration on the retention ring 120 that may be engaged into a holding fixture 117 so that forces on plug 106 are not transferred to bladder 102 and the fluid. FIG. 3B depicts a snap-fit connection 119 on a first interior location 121 of the retention ring 120 and an absent mechanical feature at a second interior location 123 of the retention ring 120. In the instance of threads, ultra-sonic welding, or adhesive bonding, no mechanical feature would be required on plug 106. The bevel and slot configuration of FIG. 3A would be required for a snap-fit.

In one embodiment, the plug 106 is secured within the tube 124 of the fill port 104 using a press-fit connection. The press-fit connection comprises cooperating members that are attached to the plug 106 and the fill port 104 and facilitate the attachment of the plug 106 to the fill port 104 by simply pressing the cylindrical member 132 of the plug 106 into the bore 126 of the fill port 104 by hand.

In one exemplary embodiment of the press-fit connection, the cooperating members comprise a tab 136 and a retention recess 138, as shown in FIGS. 4 and 5. In one embodiment, the tab 136 is attached to the plug 106 and the retention recess 138 is formed in the interior wall 128 of the tube 124 of the fill port 104, as shown in FIGS. 3 and 4. In another embodiment, the tab 136 is attached to the interior wall 128 of the fill port 104 and the retention recess 138 is formed in the cylindrical member 132 of the plug, as shown in FIG. 5.

One embodiment of the tab 136 comprises a member that extends radially from a surface 140 of the plug 106 or the interior wall 128 of the fill port 104 relative to the axis 134. In one embodiment, the tab 136 includes an annular tab that extends around the cylindrical member 132 or the interior wall 128. The annular tab can be coaxial to either the cylindrical member 132 of the plug 106 or the bore 126 of the tube 124. Another embodiment of the tab 136 comprises one or more prongs extending radially from the cylindrical member 132 or the interior wall 128.

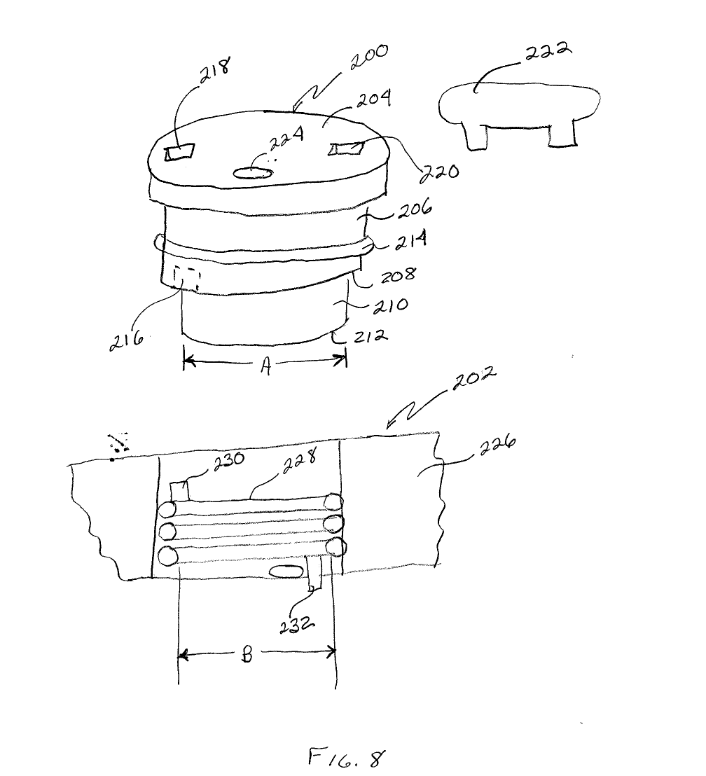

One embodiment of the retention recess 138 comprises an annular chamber formed in the interior wall 128 or the cylindrical member 132, as respectively shown in FIGS. 4 and 5. The annular chamber can be coaxial to either the cylindrical member 132 of the plug 106 or the bore 126 of the tube 124. Alternatively, the retention recess 138 may not extend entirely around the interior wall 128 or the cylindrical member 132, but can include multiple channels each configured to receive one or more tabs 136 in the form of separate prongs. The channels can be configured to allow the plug 106 to be rotate while the cylindrical member 132 is secured within the tube 124 of the fill port 104. This embodiment of the press-fit connection is particularly useful with the embodiment of the testicular prosthesis shown in FIG. 7, which will be discussed below.

A seal is formed between the cylindrical member 132 of the plug 106 and the interior wall 128 of the fill port 104. In one embodiment, a medical grade silicon adhesive or sealant is used to fill caps between the cylindrical member 132 and the interior wall 128 of the fill port 104.

In another embodiment, one or more o-rings are used to form the seal between the plug 106 and the fill port 104 as shown in FIG. 3. In one embodiment, the interior wall 128 of the cylindrical tube 124 includes an o-ring receiving recess 142, as shown in FIG. 4. One embodiment of the o-ring receiving recess 142 includes an annular recess that receives a portion of an o-ring 144. The o-ring receiving recess 142 operates to secure the o-ring 144 in position during insertion of the plug 106 into the fill port 104. In another embodiment, the o-ring receiving recess 142 is formed in the cylindrical member 132 of the plug 106, as illustrated in FIG. 5.

One embodiment of the plug 106 includes a cap 146, shown in FIG. 3. The cap 146 overhangs that cylindrical member 132 and a portion of the tube 124 of the fill port 104. The cap 146 operates to limit the distance in which the cylindrical member 132 can be inserted within the tube 124 of the fill port 104.

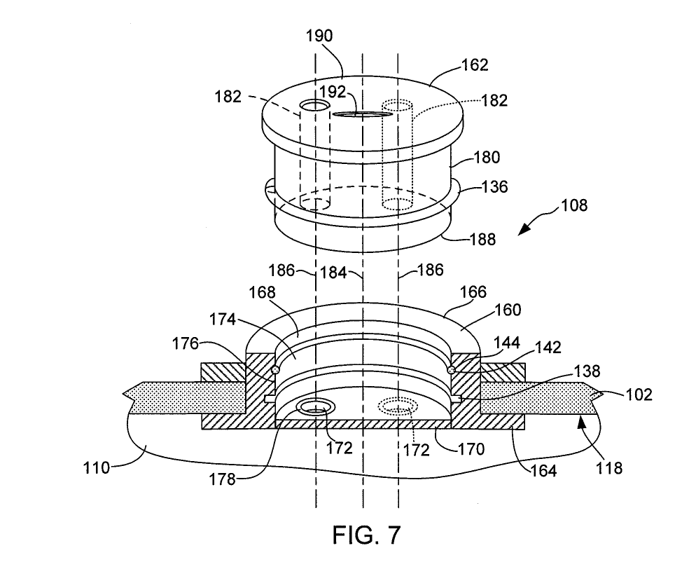

FIG. 6 illustrates a method of filling the bladder 102 with the fluid (i.e., liquid or gel) to place the bladder 102 in the inflated state. In general, a fill port plug 150 is used in place of the plug 106 described above. The fill port plug 150 is inserted into the bore of the tube 124 of the fill port 104. A seal between the fill port plug 150 and the interior wall 128 of the fill port 104 can be formed as described above, such as using an o-ring 144 within an o-ring retention recess 142, a medical grade silicon adhesive or sealant, or other manner. Additionally, the fill port plug 150 can be held in position within the bore 126 of the fill port 104 through pressure applied by the person filling the bladder 102, or using the press-fit connection described above that includes the tab 136 and the retention recess 138, as shown in FIG. 6, for example.

One embodiment of the fill port plug 150 includes a standard interface 152 for connecting to a syringe 154 or other device, as illustrated in FIG. 6. The syringe 154 can be filled with the desired fluid or gel to be injected into the interior chamber 110 of the bladder 102, as illustrated by arrow 156, to fill the interior chamber 110 with the fluid and place the bladder 102 in the inflated state. Following completion of the filling of the bladder 102 with the fluid, the fill port plug 150 can be removed from the fill port 104 and the plug 106 is inserted into the fill port 104 to seal the opening 130 and maintain the testicular prosthesis 100 in the inflated state, as illustrated in FIG. 3.

FIG. 7 illustrates a filling site 108 of the testicular prosthesis 100 in accordance with another embodiment of the invention that allows the testicular prosthesis 100 to reach an inflated state, in which the interior chamber 110 is pressurized with the fluid 112. This embodiment of testicular prosthesis 100 includes the flexible bladder 102 described above, a fill port plug receiver 160 and a fill port plug 162. The fill port plug receiver 160 is coupled to the bladder 102 in the manner described above with regard to fill port 104. For instance, the fill port plug receiver 160 can include a collar 164 that is bonded to the interior surface 118 of the bladder 102, as described above.

One embodiment of the fill port plug receiver 160 includes a housing 166 having an open top 168, a base 170 having an opening 172 and a cylindrical interior cavity 174 defined by an interior wall 176. An o-ring 178 or other sealing member is coupled to the base 170 within the interior cavity 174 and surrounds the opening 172.

One embodiment of the fill port plug 162 includes a cylindrical member 180 that can be received within the interior cavity 174 of the fill port plug receiver 160 and a fill port 182 that extends through the cylindrical member 180. The fill port plug 162 is rotatable relative to the receiver 160 between a fill position and a sealed position. Accordingly, in one embodiment, the fill port 182 is non-coaxial to a longitudinal axis 184 of the cylindrical member 180.

When the fill port plug 162 is in the fill position, the fill port 182 is aligned with the opening 172 in the base 170, as indicated by central axis 186 of the fill port 182 shown in FIG. 7. The fill position allows fluid or gel to be injected into the interior cavity 110 of the bladder 102 through the fill port 182 using a syringe or other suitable device.

The injection of fluid into the cavity 110 of the bladder 102 can be under pressure. Once the bladder 102 reaches the desired pressurized inflated state, the fill port plug 162 can be rotated about the axis 184 relative to the receiver 160 to place the plug 160 the sealed position.

When the fill port plug 162 is in the sealed position, the fill port 182 is not aligned with the opening 172 in the base 170 of the receiver 160, as indicated by the phantom depiction of the fill port 182 and opening 172 in FIG. 7. Additionally, when the fill port plug 162 is in the sealed position, the opening 172 in the base 170 of the receiver 160 is sealed through engagement between the o-ring 178 and a bottom surface 188 of the cylindrical member 180. The o-ring 178 is compressed between the base 170 and the bottom surface 188 to form the desired seal due to the coupling of the plug 162 to the receiver 160.

In one embodiment, this coupling of the plug 162 to the receiver 160 is accomplished through the press-fit connection described above. Thus, the cylindrical member 180 of the plug 162 and the interior wall 176 of the housing 166 can include the tab 136 and the retention recess 138 described above, as shown in FIG. 7. Although the tab 136 and the retention recess 138 are illustrated as being formed as components of the cylindrical member 180 and the housing 166, respectively, the depicted configuration could be reversed, as described above. The press-fit coupling of the fill port plug 162 to the fill port plug receiver 160 compresses the o-ring 172 between the base 170 and the bottom surface 188 to form the desired seal.

One embodiment of the fill port plug 162 includes a cap 190 that is attached to the cylindrical member 180 opposite the bottom surface 188. The fill port 182 extends through the cap 190 and the cylindrical member 180.

In one embodiment, the cap 190 includes a slot 192 that is configured to receive a tool for applying a torque to the plug. A tool such a screwdriver or other device can be inserted into the slot 192 to assist in the rotation of the plug 162 relative to the receiver 160 to rotate the plug 162 between the fill and sealed positions. Other features can be provided on the cap 190 to assist in applying a torque to the fill port plug 162 to rotate the plug 162 about the axis 184 relative to the fill port plug receiver 160.

Another embodiment of the testicular prosthesis 100 includes a seal formed between the cylindrical member 180 of the plug 160 and the interior wall 176 of the housing 166. Embodiments of the seal include those described above with respect to fill port 104 and plug 106. For example, an o-ring 144 can be received within a retention recess 142 of either the cylindrical member 180 of the fill port plug 160 or the housing 166 (shown) of the fill port plug receiver 160 to perform the desired seal.

A further embodiment of the fill port plug and fill port plug receiver is shown in FIG. 8. In this instance a fill port plug 200 and fill port plug receiver 202 are identified. This embodiment of the fill port plug 200 includes a cap 204 that is attached to a cylindrical member 206 opposite a bottom edge 208. A further cylindrical member 210, having diameter “A” continues downward from bottom edge 208 to bottom surface 212. An o-ring 214 is provided about the circumference of cylindrical portion 206 and a tab receiver 216 is provided within cylindrical portion 206. The cap 204 is provided with a pair of tool slots 218, 220 in a configuration to receive a tool, such as a spanners wrench 222. Fill port 224, which extends the length of fill port plug, is also depicted.

Fill port plug receiver 202, shown within housing 226, incorporates a torque spring 228 that provides a first spring tab 230 and a second spring tab 232. The torque spring 228 is defined by a diameter “B” that is of sufficient diameter to receive cylindrical member 210, which is defined by diameter “A”. The first spring tab 230 is designed to engage tab receiver 216 while second spring tab 232 is designed to engage housing 226. A reservoir port 234 is provided.

To operate the embodiment of FIG. 8, spanner wrench 222 is placed within tool slots 218, 220. Spanner wrench 222 is then rotated to turn fill port plug 200 from a normally closed position to an open position. Then, while continuing to hold wrench 222, fill port 224 is aligned with reservoir port 234. A needle stub (not shown) can then be placed through fill port 224 into reservoir port 234. At this point wrench 222 can be removed as the needle will act to hold fill port plug 202 in its open position. The needle syringe can be depressed to force fluid into the prosthesis. Upon removal of the needle stub from fill port plug 224, fill port plug 224 rotates back to its normally closed position under direction of torque spring 228.

Although the present invention has been described with reference to preferred embodiments, workers skilled in the art will recognize that changes may be made in form and detail without departing from the spirit and scope of the invention.

What is claimed is:

1. A testicular prosthesis implantable within a patient for the purpose of replacing a natural testicle, the testicular prosthesis comprising:
a flexible bladder having an interior chamber;
a fill port coupled to the flexible bladder, the fill port comprising a cylindrical tube extending through the bladder and having a cylindrical bore defined by an interior wall and forming an opening to the interior chamber of the bladder;
a plug comprising a cylindrical member that is coaxial to the bore; and
a seal formed between the cylindrical member of the plug and the interior wall of the fill port.

2. The prosthesis of claim 1, wherein:
the interior wall of the cylindrical tube comprises an o-ring receiving recess; and
the seal comprises an o-ring received in the o-ring receiving recess.

3. The prosthesis of claim 1, wherein:
the exterior surface of the cylindrical member comprises an o-ring receiving recess; and
the seal comprises an o-ring received in the o-ring receiving recess.

4. The prosthesis of claim 1, wherein the plug further comprises a cap, from which the cylindrical member extends, wherein the cap overhangs the cylindrical member and a portion of the tube of the fill port.

5. The prosthesis of claim 1, wherein the fill port further comprises an annular collar that is coupled to the tube, coaxial to the bore and coupled to the bladder, the collar forming a seal between the bladder and the fill port.

6. The prosthesis of claim 5, wherein the collar is attached to an interior surface of the bladder.

7. The prosthesis of claim 6, further comprising a retention ring, through which the tube extends, the retention ring positioned on an exterior surface of the bladder opposite the collar.

8. The prosthesis of claim 1, further comprising a press-fit connection comprising:
a tab extending radially from the cylindrical member of the plug; and
a retention recess formed in the interior wall of the fill port, wherein the tab is received within the retention recess.

9. The prosthesis of claim 1, further comprising a press-fit connection comprising:
a tab extending radially from the interior wall of the fill port; and
a retention recess formed in the cylindrical member of the plug, wherein the tab is received within the retention recess.

10. The prosthesis of claims 8 or 9, wherein the retention recess is annular and coaxial to either the bore of the tube or the cylindrical member of the plug.

11. The prosthesis of any of claims 8-10, wherein the tab is annular and coaxial to either the bore of the tube or the cylindrical member of the plug.

12. A testicular prosthesis implantable within a patient for the propose of replacing a natural testicle, the testicular prosthesis comprising:
a flexible bladder having an interior chamber;
a fill port plug receiver coupled to the flexible bladder comprising:
a housing having an open top, a base having an opening and a cylindrical interior cavity defined by an interior wall; and
a first o-ring coupled to the base within the interior cavity and surrounding the opening;
a fill port plug comprising:
a cylindrical member received within the interior cavity of the fill port plug receiver; and
a fill port extending through the cylindrical member; and
wherein the fill port plug is rotatable within the receiver between a fill position, in which the fill port is aligned with the opening in the base of the receiver, and a sealed position, in which the opening in the base of the receiver is sealed through engagement between the first o-ring and a bottom surface of the cylindrical member.

13. The prosthesis of claim 12, wherein the fill port plug comprises a cap attached to the cylindrical member opposite the bottom surface, wherein the fill port extends through the cap.

14. The prosthesis of claim 13, wherein the cap includes a slot configured to receive a tool for applying a torque to the plug.

15. The prosthesis of claim 12, wherein the fill port is non-coaxial to a longitudinal access of the cylindrical member.

16. The prosthesis of claim 12, further comprising a seal formed between the interior wall of the housing and the exterior surface of the cylindrical member.

17. The prosthesis of claim 16, wherein:
the interior wall of the housing comprises an o-ring receiving recess; and
the seal comprises a second o-ring received in the o-ring receiving recess.

18. The prosthesis of claim 16, wherein:
the exterior surface of the cylindrical member comprises an o-ring receiving recess; and
the seal comprises a second o-ring received in the o-ring receiving recess.

19. The prosthesis of claim 12, wherein the fill port plug receiver further comprises a collar coupled to the housing in the bladder, the collar forming a seal between the bladder and the housing.

20. The prosthesis of claim 12, wherein the collar is attached to an interior surface of the bladder.

21. The prosthesis of claim 20, further comprising a retention ring, through which the housing extends, the retention ring positioned on an exterior surface of the bladder opposite the collar.

22. The prosthesis of claim 12, further comprising a press-fit connection comprising:
a tab extending radially from an exterior surface of the cylindrical member of the plug; and
a retention recess formed in the interior wall of the fill port plug receiver, wherein the tab is received within the retention recess.

23. The prosthesis of claim 12, further comprising a press-fit connection comprising:
a tab extending radially from the interior wall of the fill port plug receiver; and
a retention recess formed in the cylindrical member of the plug, wherein the tab is received within the retention recess.

24. The prosthesis of claims 22 or 23, wherein the retention recess is annular and coaxial to either the bore of the tube or the cylindrical member of the plug.

25. The prosthesis of any of claims 22-24, wherein the tab is annular and coaxial to either the bore of the tube or the cylindrical member of the plug.
